(19) United States
(12) Patent Application Publication
(10) Pub. No.: US 2024/0150418 A1
(43) Pub. Date: May 9, 2024

(54) IMMUNOGENIC PROTEIN HYDROLYSATE WITH REDUCED ALLERGENICITY

(71) Applicant: Arla Foods amba, Viby J (DK)

(72) Inventors: Hans Peter Sørensen, Viby J (DK); Hans Bertelsen, Viby J (DK); Søren Klitgaard, Viby J (DK); Lotte Neergaard Jacobsen, Viby J (DK); Ditte Møller Nielsen, Viby J (DK)

(21) Appl. No.: 18/547,133

(22) PCT Filed: Mar. 2, 2022
(86) PCT No.: PCT/EP2022/055289

§ 371 Date: Aug. 18, 2023

(30) Foreign Application Priority Data
Mar. 2, 2021 (EP) 21160297.4
Mar. 25, 2021 (EP) 21164975.1

(51) Int. Cl. C07K 14/47; A61P 37/08; C12N 9/64; C12P 21/06; A61K 38/00

(52) U.S. Cl. CPC C07K 14/47; A61P 37/08; C12N 9/6408; C12N 9/6472; C12N 9/6489; C12P 21/06; A61K 38/00; C12Y 304/21062; C12Y 304/22039; C12Y 304/24027

(57) ABSTRACT

The present invention relates to a new whey protein hydrolysate having reduced allergenicity and retained immunogenicity. The new whey protein hydrolysate is also characterized by having a low amount of larger peptides. The invention furthermore relates to a method of preparing the new whey protein hydrolysates, uses of the new whey protein hydrolysates and food products comprising this new whey protein hydrolysate.

### TECHNICAL FIELD OF THE INVENTION

The present invention relates to cow's milk allergy and induction of oral tolerance. In particular, the present invention relates to a whey protein hydrolysate with reduced allergenicity and a retained immunogenicity and a method of preparing said whey protein hydrolysate.

### BACKGROUND OF THE INVENTION

Food allergy is an abnormal immune response that occurs after eating a certain food. In general, allergy results from the failure to develop immune tolerance to harmless proteins, termed allergens. Instead, an immune response occurs—known as sensitization. Once sensitized, the individual may experience an adverse reaction (clinical allergy) on renewed exposure to a sufficient dose of the allergen. The allergic reaction typically occurs within minutes to hours after exposure, and the symptoms range from mild (lip swelling, itching in the mouth) to severe. In some people it can even cause life-threatening reactions (anaphylaxis). Allergy manifests in a variety of symptoms including skin, respiratory and gastrointestinal reactions.

About 6-8% of children under the age of three are estimated to be affected by food allergy. Cow's milk is one of the most frequent causes of food allergy in children with a prevalence of 2-3%.

Allergic diseases are increasing globally, hence, the need for effective preventive solutions is increasing as well. Prevention is crucial as there remains a strong trend in infants suffering and recovering from cow's milk allergy to develop atopic symptoms such as atopic dermatitis, asthma and hay fever later in life. This is the so-called “allergic march”, which is a burden in terms of later illness, quality of life, and health care costs. Today no cure exists and the best way of preventing an allergic reaction is to avoid the food identified as the allergen.

As allergy to cow's milk is a major problem worldwide, and as cow's milk allergy has a high prevalence in infants, there is a huge need for infant formulas preventing the development of allergy.

One of the mechanisms used by the body to avoid an allergic reaction is to build up tolerance (prevent sensitization) for a given substance. Tolerance is induced by substances having a high immunogenicity. Oral tolerance is the active non-response of the immune system to an allergen administered through the oral route. The mucosal immune system is capable of distinguishing between harmful and harmless compounds resulting in inflammation or tolerance, respectively. Food components are important non-self allergens to which an immune response constantly needs to be suppressed. This type of tolerance induction is known as oral tolerance. It is increasingly evident that allergen avoidance may be unsuccessful or even detrimental in allergy prevention. Induction of oral tolerance by peptides derived from food proteins is a powerful measure to drag the immune system towards tolerance instead of sensitization

It is well known to use whey protein hydrolysates as ingredients in various food products including infant formulas. Whey protein hydrolysates are commonly prepared by hydrolysing a whey protein substance, such as a whey protein concentrate, with a food grade proteolytic and/or peptidolytic preparation to a desired degree of hydrolysis. In some situations, it is desired to prepare a whey protein hydrolysate with a low amount of large peptides, and a high degree of hydrolysis in order for the whey protein hydrolysates to have a low allergenicity.

Some existing whey protein hydrolysates used for allergy management have been subjected to ultrafiltration. Ultrafiltration will remove larger peptides, and such ultrafiltered whey protein hydrolysates will not induce an allergic response. However, even though ultrafiltered whey protein hydrolysates have low allergenicity, which means an allergic response is avoided when administered to infants or children allergic to cow's milk, a drawback is that they also have a low immunogenicity. Hence, ultrafiltered whey protein hydrolysates do not allow induction of oral tolerance to the cow's milk protein.

In for example EP 0 642 307 A1, the inventors use ultrafiltration following hydrolysis in a method of producing a whey protein hydrolysate having a degree of hydrolysis of 15% to 35%. As disclosed in EP 0 642 307 A1, the whey protein hydrolysate obtained has reduced allergenicity. However, because of the ultrafiltration step, the immunogenicity will in addition be low, which might prevent oral tolerance induction.

Further, in the art there has been a desire to produce a whey protein hydrolysate with a high solubility and heat stability. This can be achieved by adding minerals to the product during the hydrolysis. However, due to local restrictions in different parts of the world, it is a requirement to limit the amount of added minerals, including ash, to the product. In China, the content of ash has to be limited to 5.5% or less.

Hence, a whey protein hydrolysate having a low fraction of large peptides (peptides above 2500 Da) and therefore a reduced allergenicity, without decreasing the immunogenicity would be advantageous.

### SUMMARY OF THE INVENTION

The inventors of the present invention have surprisingly found that by using a specific two-step method of hydrolysis with heat treatment in-between the hydrolysis steps, combined with a specific combination of enzymes for enzymatic hydrolysis of whey protein, it is possible to obtain whey protein hydrolysates with a low amount of large peptides (less than 7.5% peptides with a molecular weight larger than 2500 Da of the total protein content), combined with reduced allergenicity and retained immunogenicity. In addition, the whey protein hydrolysate comprises a low amount of ash.

The heat treatment between the two hydrolysis steps causes unfolding of the proteins. This unfolding exposes buried parts of the protein and gives the enzymes in the second step of hydrolysis access to new cleavage sites that before the heat treatment were inaccessible. Hereby, further hydrolysis of the protein is obtained, and the protein hydrolysate obtained comprises a lower fraction of large peptides as compared to protein hydrolysates prepared without the heat treatment.

An object of the present invention relates to a whey protein hydrolysate having a reduced allergenicity and a retained immunogenicity.

One aspect of the present invention relates to a whey protein hydrolysate having the following characteristics:
i) comprising peptides with a molecular weight of 2500 Da or above in an amount of 7.5% by weight of the total protein content or less;
ii) an allergenicity corresponding to a concentration of beta-lactoglobulin (BLG) being less than 500 mg BLG/kg protein measured in an RBL cell assay;
iii) an immunogenicity based on the serum IgG titre of immunised animals that is essentially the same as for non-hydrolysed whey protein;
iv) comprising essentially no intact protein.

Another aspect of the present invention relates to a method of preparing a whey protein hydrolysate comprising the steps of:
a) providing a solution comprising whey protein;
b) adding at least a subtilisin from Bacillus licheniformis, a thermolysin from Bacillus amyloliquefaciens and a cysteine endoprotease from Ananas comosus to the solution comprising whey protein and performing a first hydrolysis step;
c) heat-treating the hydrolysed solution of step b) by adjusting the temperature to at least 60° C. and maintaining the temperature at at least 60° C. for a period of time sufficient to unfold remaining folded protein;
d) adjusting the temperature of the heat-treated solution of step c) to a temperature of 50° C. to 70° C.;
e) adding at least a subtilisin from Bacillus licheniformis to the hydrolysate of step d) and performing a second hydrolysis step;
f) inactivating the enzymes when the degree of hydrolysis is at least 17%; to obtain a whey protein hydrolysate.

In a further aspect, the invention relates to a food product comprising the whey protein hydrolysate according to the invention.

In still a further aspect, the invention relates to the use of the whey protein hydrolysate according to the present invention in nutrition for infants.

Yet another aspect of the present invention relates to the whey protein hydrolysate according to the invention, for reducing the risk of allergy to cow's milk proteins in infants or patients in need thereof.

In an even further aspect, the invention relates to the whey protein hydrolysate according to the invention, for prevention of or reducing the risk of developing atopic dermatitis, asthma, and/or allergic rhinitis in infants or patients in need thereof.

### BRIEF DESCRIPTION OF THE FIGURES

FIG. 1 shows a HPLC chromatogram of a sample of the whey protein hydrolysate of the present invention for detection and quantification of beta-lactoglobulin and alpha-lactalbumin.

FIGS. 2A, B and C show the IgG1 titre in rat serum on day 21, 28 and 35 respectively of rats immunized with different samples of non-hydrolysed and hydrolysed whey protein.

FIGS. 3A, B and C show IgE titre in rat serum on day 21, 28 and 35 respectively of rats immunized with different samples of non-hydrolysed and hydrolysed whey protein.

The present invention will now be described in more detail in the following.

### DETAILED DESCRIPTION OF THE INVENTION

### Definitions

Prior to discussing the present invention in further details, the following terms and conventions will first be defined:

All references to singular characteristics or limitations of the present invention shall include the corresponding plural characteristic or limitation, and vice versa, unless otherwise specified or clearly implied to the contrary by the context in which the reference is made.

All percentages referred to herein are percentages by weight unless otherwise stated. Also, the terms “by weight of dry matter” and “on dry matter basis” refer to the same concept and are used interchangeably.

The term “w/w” as in for example 1% w/w refers to a composition comprising 1% by weight of a compound.

In the context of the present invention, the term “beta-lactoglobulin” may also be referred to as “BLG”. The terms may be used interchangeably and pertain to BLG from mammal species. Furthermore, the term “alpha-lactalbumin” may in the context of the present invention be referred to as “ALA” and pertains to alpha-lactalbumin from mammal species.

Whey Protein Hydrolysate

In an aspect, the present invention relates to a whey protein hydrolysate having the following characteristics:
i) comprising peptides with a molecular weight of 2500 Da or above in an amount of 7.5% by weight of the total protein content or less;
ii) an allergenicity corresponding to a concentration of beta-lactoglobulin (BLG) being less than 500 mg BLG/kg protein measured in an RBL cell assay;
iii) an immunogenicity based on the serum IgG titre of immunised animals that is essentially the same as for non-hydrolysed whey protein;
iv) comprising essentially no intact protein.

Hence, the present invention relates to a whey protein hydrolysate that has a low amount of peptides with a high molecular weight, a reduced allergenicity and a retained immunogenicity and essentially no intact protein present.

The term “allergenicity” refers to a substance being an allergen. An allergen is a type of antigen inducing an unwanted immune response towards something otherwise harmless to the body. The allergen is capable of stimulating a reaction through IgE responses by the immune system in the subject. IgE is under normal conditions directed towards defending the body against infection.

The term “allergenic potential” refers to a substance with antibody binding capabilities. Binding is a prerequisite for an allergic reaction to happen. The allergenic potential may be determined using an ELISA assay.

In connection with the present invention, the term “sensitization” is also used. Immune responses in allergy begin with sensitization. The first exposure to a sensitizing allergen will stimulate plasma B-cells to produce IgE-antibodies, which, like antibodies of other immunoglobulin isotypes, are capable of binding a specific allergen via its Fragment antigen binding (Fab) portion. Different allergens stimulate the production of corresponding allergen-specific IgE antibodies. In cow's milk, beta-lactoglobulin is an allergen of concern and the immune system may mediate allergic reactions by clonal selection of antibodies reacting towards specific epitopes on beta-lactoglobulin. Once formed and released into the circulation, IgE binds, through its Fragment crystallizable (Fc) portion, to high affinity receptors on mast cells, leaving its allergen-specific receptor site (the paratope) available for future interaction with the allergen (e.g. epitopes on beta-lactoglobulin). Other cells known to express high-affinity receptors for IgE include basophils, Langerhans cells and activated monocytes. Production of allergen-specific IgE antibodies completes the immune response known as sensitization. After re-exposure to the same allergen, the allergen can bind and cross-link the specific IgE antibodies on the surface of the mast cells, resulting in degranulation of the mast cells and release of inflammatory mediators such as histamine, leading to allergic reactions.

The term “whey protein hydrolysate” refers in the context of the present invention to the same that the skilled person knows as a whey protein hydrolysate, namely a hydrolysed whey protein material obtained by enzymatic hydrolysis of a whey protein starting material. Hence, a whey protein hydrolysate is a product obtained directly by enzymatic hydrolysis of a whey protein starting material. Hence, any composition that arbitrary combines peptides (even though they may come from whey) are not a whey protein hydrolysate if the composition is not obtained by enzymatic hydrolysis of whey protein.

The whey protein hydrolysate of the present invention is characterized by having a reduced allergenicity. A reduced allergenicity can be expressed based on the concentration of beta-lactoglobulin, since the whey protein, beta-lactoglobulin, is an allergen and therefore may cause an allergic response in individuals allergic to cow's milk. A non-hydrolysed whey protein concentrate may comprise beta-lactoglobulin in an amount of about 540,000 mg/kg powder. However, after protease treatment, beta-lactoglobulin is hydrolysed into peptides, and the concentration of beta-lactoglobulin in the whey protein hydrolysate that can cause an allergic response is therefore reduced. Hence, when expressing allergenicity of whey protein hydrolysates, the concentration of beta-lactoglobulin can be used. In the whey protein hydrolysate of the present invention, it was found that the concentration of beta-lactoglobulin was 500 mg/kg total protein or less measured in an RBL (rat basophilic leukemia) cell assay.

The allergenicity of the whey protein hydrolysate of the invention is expressed by the degranulation of IgE antibodies by a protein preparation such as a protein hydrolysate.

Degranulation is a consequence of cross-linking of food allergens by specific IgE antibodies on effector cells like basophils/mast cells. For degranulation to happen, the allergen needs to be large enough to include two different epitopes and thereby be bound by two IgE antibodies on the basophil/mast cell. When an allergen is bound to two IgE antibodies attached to the cell it causes cross-linking of IgE antibodies leading to a degranulation, which will release mediators causing a variety of symptoms in the subject. A non-limiting example of such an allergen is beta-lactoglobulin.

Thus, in an aspect of the present invention the whey protein hydrolysate has an allergenicity corresponding to less than 500 mg BLG/kg protein measured in an RBL cell assay. RBL cell assays are well known in the art and RBL cells are commonly known to to be used in allergy studies due to the strong response of the cells to IgE and its FcεRI receptor, see for example Na Sun et al “Use of a rat basophil leukemia (RBL) cell-based immunological assay for allergen identification, clinical diagnosis of allergy, and identification of anti-allergy agents for use in immunotherapy”, Journal of Immunotoxicology, p. 199-2052014. Different RBL assays could be used in connection with measuring allergenicity of the whey protein hydrolysate of the present invention, an example is a RBL assay provided by Polpharma Biologics S.A., Poland.

In the context of the present invention, the term “beta-lactoglobulin” or (BLG) refers to the major protein in whey of cow's milk. Whey BLG represents a well-known allergen and inducer of IgE responses. Thus, it is possible to use the level of beta-lactoglobulin as an indication of the potential of a substance to mediate allergenicity.

In an embodiment of the invention, the whey protein hydrolysate has an allergenic potential corresponding to a concentration of beta-lactoglobulin being 500 mg/kg total protein or less measured using an enzyme-linked immunosorbent assay (ELISA). ELISA assays are well known in the art and any type of ELISA assays could be used to measure BLG. An example of an ELISA assay that could be used to measure the beta-lactoglobulin concentration is a competitive enzyme immunoassay, i.e. competitive ELISA assay. This could for example be the ELISA assay RIDASCREEN® beta-lactoglobulin assay (Art. No. R4901) provided by R-Biopharm AG, Germany, which is designed to quantify native and processed residual beta-lactoglobulin in whey protein hydrolysates and food products. However, other ELISA assays could be used to measure the BLG concentration.

In a preferred embodiment of the invention, the allergenic potential is corresponding to a concentration of beta-lactoglobulin being 400 mg/kg total protein or less measured in an ELISA, preferably the allergenic potential is corresponding to a concentration of beta-lactoglobulin being 350 mg/kg total protein or less measured in an ELISA, even more preferably allergenic potential is corresponding to a concentration of beta-lactoglobulin being 300 mg/kg total protein or less measured in an ELISA.

Another way of expressing allergenicity in a whey protein hydrolysate is by the IgE antibody titre in serum from animals subjected to the protein hydrolysate. The term “IgE titre” is a measure of the concentration of IgE antibodies in serum from animals subjected to the protein hydrolysate. Here, antibody titre (IgG or IgE) is defined as the concentration of antibody in rat serum that recognizes epitopes from whey proteins, expressed as the inverse of the highest dilution that gives a positive result in an ELISA.

Allergenicity can be defined as “a hypersensitivity reaction mediated by immunological mechanisms” which can be antibody- or cell-mediated. In the majority of cases, the antibody typically responsible for an allergic reaction belongs to the IgE isotype and individuals may be referred to as suffering from an IgE-mediated allergic disease. Therefore, measuring the IgE antibody titre in serum from animals subjected to the protein hydrolysate is a good way of expressing the level of IgE-mediated allergenicity.

Non-IgE mediated allergies are caused by a reaction involving other components of the immune system apart from IgE antibodies. The reactions do not appear immediately after the ingestion of the food and usually relate to reactions in the gastrointestinal tract. By contrast the signs and symptoms of IgE mediated food allergy usually occur within minutes of ingestion and include hives, redness of the skin, vomiting and in more severe reactions, anaphylaxis. Unlike IgE mediated food allergy there are no blood or skin tests which have proved useful in general medical practice to diagnose non-IgE mediated food allergy, as the mechanisms of this type of food allergy is not well understood.

It is assumed that food allergic individuals are equally divided into IgE-mediated and non-IgE mediated allergy. However, both types of allergy can be seen in the same individual.

In an embodiment of the invention, the whey protein hydrolysate provides an IgE antibody titre in serum after 35 days from animals subjected to the protein hydrolysate being at most 50% of the IgE antibody titre obtained in serum after 35 days from animals subjected to the same concentration of intact whey protein. The animal is preferably a mammal. In the examples describing the invention, rats have been used to determining the IgE antibody titre. Preferably, the IgE antibody titre in serum after 35 days from the animals are subjected to the protein hydrolysate is at most 40% of the IgE antibody titre obtained in serum after 35 days from animals subjected to the same concentration of intact whey protein

The whey protein hydrolysate of the present invention is also characterized by having a retained immunogenicity, which can be expressed by the IgG antibody titre in serum from animals subjected to the protein hydrolysate. The term “titre” refers to the presence and amount of antibodies in serum from animals subjected to the protein hydrolysate. IgG is produced as the primary and secondary responses to antigens such as beta-lactoglobulin. Therefore, the concentration of IgG towards a specific antigen in serum is a measure of the exposure of the immune system to this antigen and also the ability of the antigen to stimulate the immune system (immunogenicity).

The term “immunogenicity” refers to the ability of a foreign substance to induce an immune response in the subject. In food allergy, this is seen as a controlled immune reaction to allergens like beta-lactoglobulin. The response is mediated by production of specific IgG antibodies.

In the present invention, the inventors have discovered a process to produce a protein hydrolysate where the immunogenicity can be maintained while the allergenicity is decreased.

Hence, in an aspect of the invention, the whey protein hydrolysate is having an immunogenicity based on the serum IgG titre of immunised animals that is essentially the same as for immunised animals with non-hydrolysed whey protein.

The immunogenicity based on the IgG titre of immunised animals may for example be measured by using the Brown Norway rat model as described in the article “Preclinical Brown Norway Rat Models for the Assessment of Infant Formulas in the Prevention and treatment of Cow's Milk Allergy”, Jensen et al., published in International Archives of Allergy and Immunology, 13 Feb. 2019, DOI: 10.1159/000495801 and the article “Characterization of the Immunogenicity and Allergenicity of Two Cow's Milk Hydrolysates—A Study in Brown Norway Rats”, Bøgh et al., published in Scandinavian Journal of Immunology, 23 Dec. 2014, DOI: 10.1111/sji.12271.

The term “essentially the same” is in the context of the present invention referred to as the serum IgG titre of animals immunised with the whey protein hydrolysate and the IgG titre of animals immunised with non-hydrolysed whey protein are not significantly different. Another way to define the term “essentially the same” is that the difference between the serum IgG titre of an animal immunised with the whey protein hydrolysate and the serum IgG titre of an animal immunised with non-hydrolysed whey protein is not more than 5%, such as not more than 4%, and preferably not more than 3%.

The inventors of the present invention surprisingly found that with the method of the invention, it was possible to obtain a whey protein hydrolysate having a low amount of peptides with a molecular weight of 2500 Da or above in an amount of 7.5% by weight of the total protein content or less, and that said hydrolysate had a reduced allergenicity combined with a retained immunogenicity. With the protein hydrolysate of the invention, it is therefore possible for the immune system to detect the hydrolysate and oral tolerance may be induced. The inventors believe that said hydrolysate with a retained immunogenicity and a reduced allergenicity may lead to oral tolerance induction without adverse allergic reactions, when administered to an animal. The animal is preferably a mammal.

An object of the present invention was to make a whey protein hydrolysate with a high degree of hydrolysis, which without any step of ultrafiltration had a reduced allergenicity combined with a retained immunogenicity.

It was an object of the invention to prepare a whey protein hydrolysate having reduced allergenicity while retaining the immunogenicity and at the same time have a small amount of larger peptides. It was surprisingly found by the inventors of the present invention that with the method of the present invention it was possible to obtain this. It was particularly surprising for the inventors that with the method of the present invention, it was not only possible to obtain a whey protein hydrolysate having reduced allergenicity and retained immunogenicity, but it was also possible to obtain a whey protein hydrolysate having a small amount of larger peptides, i.e. 7.5% by weight peptides of the total protein content or less having a molecular weight of 2500 Da or more. Further, it was surprising for the inventors of the present invention that a whey protein with said characteristics could be obtained which also had a high degree of hydrolysis.

The whey protein hydrolysate of the present invention typically comprises more than 70% by weight and less than 80% by weight of the total protein content having a molecular weight of 375 to 2500 Da. Further, more than 55% by weight and less than 68% by weight of the total protein content in the whey protein hydrolysate of the invention is having a molecular weight of 375 to 1250 Da. In addition, the whey protein hydrolysate of the present invention comprises 15% to 20% by weight of the total protein content having a molecular weight of 375 Da or less.

Hence, in a preferred embodiment of the invention, the degree of hydrolysis of the whey protein hydrolysate is at least 17%, even more preferably, the degree of hydrolysis of the whey protein hydrolysate is at least 20%.

In another embodiment of the present invention, the whey protein hydrolysate according to the invention has a degree of hydrolysis from 17 to 30%, such as from to 25%.

In an aspect of the invention, the whey protein hydrolysate comprises essentially no intact protein. Hence, the whey protein hydrolysate comprises essentially no non-hydrolysed protein. The terms “intact protein” and “non-hydrolysed protein” refer to the same. In an embodiment, the whey protein hydrolysate comprises essentially no intact whey protein, preferably the whey protein hydrolysate comprises essentially no intact beta-lactoglobulin and no intact alpha-lactalbumin.

The term “essentially no intact protein” refers to the amount of intact protein, such as whey protein, beta-lactoglobulin and alpha-lactalbumin, in the whey protein hydrolysate is 1.0% by weight or less of the total protein content, such as 0.5% by weight or less of the total protein content. The term “total protein content” refers to the total amount of proteins and peptides derived from proteins.

The whey protein hydrolysate may comprise other ingredients than protein, for example carbohydrates, lipids and minerals.

In an embodiment of the invention, the whey protein hydrolysate comprises carbohydrates in an amount of 10% by weight or less based on the total solid content, such as 5% by weight or less based of the total solid content, and preferably in an amount of 4% by weight or less based on the total solid content.

In an embodiment of the invention, the whey protein hydrolysate comprises lipids in an amount of 15% by weight or less based on the total solid content, such as 10% by weight or less based on the total solid content, preferably 8% by weight or less based on the total solid content. In another embodiment, the whey protein hydrolysate comprises lipids in an amount of 1% by weight or less based on the total solid content, such as a lipid content of 0.5% by weight or less of the total solid content.

It was surprisingly found by the inventors of the present invention that the whey protein hydrolysate obtained by the method of the invention could be prepared while retaining a low ash content in the protein hydrolysate.

Hence, in one embodiment of the present invention, the whey protein hydrolysate comprises ash in an amount of 6.0% by weight or less of the solid content, such as in an amount of 5.0% by weight or less of the solid content. A powder of whey protein hydrolysate according to the present invention comprises preferably 94-95% by weight of solid content. Therefore, the whey protein hydrolysate as such comprises ash in an amount of 5.5% by weight or less, such as 5.0% by weight or less.

Ash can for example be measured by using the GB5009.4-2016 Chinese standard for measuring ash content in food products. This Standard is a national Food Safety Standard of the People's Republic of China National Food Safety Standard for determining the ash content in foods.

In a preferred embodiment of the invention, the whey protein hydrolysate is in the form of a dry composition, such as a powder or granulate, preferably a powder.

In another embodiment, the whey protein hydrolysate is a liquid composition.

Method for Producing the Whey Protein Hydrolysate:

In an aspect, the present invention relates to a method of preparing a whey protein hydrolysate comprising the steps of:
a) providing a solution comprising whey protein;
b) adding at least a subtilisin from Bacillus licheniformis, a thermolysin from Bacillus amyloliquefaciens and a cysteine endoprotease from Ananas comosus to the solution comprising whey protein and performing a first hydrolysis step;
c) heat-treating the hydrolysed solution of step b) by adjusting the temperature to at least 60° C. and maintaining the temperature at at least 60° C. for a period of time sufficient to unfold remaining protein;
d) adjusting the temperature of the heat-treated solution of step c) to a temperature of 50° C. to 70° C.;
e) adding at least a subtilisin from Bacillus licheniformis to the hydrolysate of step d) and performing a second hydrolysis step;
f) inactivating the enzymes when the degree of hydrolysis is at least 17%; to obtain a whey protein hydrolysate.

The feed material for use in the method of the invention is a solution comprising whey protein.

Solution Comprising Whey Protein:

In the context of the present invention, the term “solution” as in “solution comprising whey protein” encompasses compositions that contain a combination of liquid and solid compounds or semi-solid particles such as protein particles. A “solution” may therefore be a suspension or even a slurry. However, the “solution” is preferably pumpable and the amount of liquid in the solution comprising whey protein is preferably 70-98%, more preferably 80-96%. The liquid used for the whey protein solution is typically water.

The solution comprising whey protein will typically comprise protein in an amount of 2% by weight or more of the solution. In an embodiment of the invention, the whey protein solution comprises protein in the range from 2% to 20% by weight of the solution. Preferably, the solution comprising whey protein comprises protein in an amount in the range of from 5% to 15% by weight of the solution.

The solution comprising whey protein used in the hydrolysis is obtained by dispersing a composition comprising whey protein in a liquid, such as water. Preferably, the solution comprising whey protein is made by mixing any of a milk serum protein concentrate, a whey protein concentrate, a milk serum protein isolate and/or a whey protein isolate with water. Thus, in an embodiment of the present invention, the solution comprising whey protein comprises a milk serum protein concentrate, a whey protein concentrate, a milk serum protein isolate and/or a whey protein isolate.

The solution comprising whey protein of the present invention comprises whey protein in an amount of at least 50% based on the total solid content. If the content of whey protein is less than 50% of the total solid content, the overall molecular composition (ratios between protein, carbohydrates, lipids and minerals) will be different, the enzymes may thus behave differently and hence the product obtained will be different.

Besides from whey protein, the whey protein solution may comprise other proteins in small amounts, for example casein.

The whey protein solution typically comprises other components in addition to protein. The whey protein solution may comprise other components that are normally found in whey or milk serum, such as minerals, carbohydrates, and/or lipids. Alternatively or additionally, the whey protein solution may comprise components that are not native in the whey or milk serum. However, such non-native milk components should be suitable and safe for use in food production.

The lower the content of protein is in the whey protein solution, based on the total solid content, the higher the amounts of lipids, carbohydrates (mainly lactose) and other proteins than whey protein are.

The solution comprising whey protein may for example comprise carbohydrates, such as lactose, oligosaccharides and/or hydrolysis products of lactose (i.e. glucose and galactose). The whey protein solution may e.g. comprise carbohydrate in the range of from 0% to 15% by weight based on the total solid content.

If a whey protein concentrate (WPC) or milk serum protein concentrate (SPC) is used for preparing the solution comprising whey protein, the amount of carbohydrates in the solution is preferably in the range of from 2 to 8% by weight based on the total solid content.

Whey protein isolate (WPI) and milk serum protein isolate (SPI) might in addition be used for preparing the solution comprising whey protein. Here, the amount of carbohydrates, such as lactose, is very low. Hence, when a WPI or SPI is used for preparation of the solution comprising whey protein, the content of carbohydrates in the solution comprising whey protein is in the range of from 0% to 1% by weight based on the total solid content.

The solution comprising whey protein may also comprise lipids, e.g. in the form of triglycerides and/or other types of lipids, such as phospholipids.

In the context of the present invention, the terms “fat” and lipid” have the same meaning and may be used interchangeably.

The solution comprising whey protein according to the present invention should comprise whey protein in an amount of at least 50% by weight based on the total solid content. If the protein content in the solution comprising whey protein is less than 50% by weight of the total solid content, the whey protein hydrolysate obtained after hydrolysis may not have the characteristics defining the whey protein hydrolysate according to the present invention.

The solution comprising whey protein may for example comprise whey protein in an amount of 50-98% by weight based on the total solid content, such as at 60-92% by weight based on the total solid content, even more preferably 65-90% by weight based on the total solid content. In a more preferred embodiment of the present invention, the solution comprising whey protein comprises whey protein in an amount of at least 80% by weight based on the total solid content.

The protein present in the solution comprising whey protein should mainly be whey proteins. However, minor amounts of other proteins, such as for example casein, may be present. In an embodiment of the present invention, the solution comprising whey protein therefore comprises whey protein in an amount of 90% by weight or more based on the total amount of protein. Preferably, the solution comprising whey protein comprises whey protein in an amount of 95% by weight or more based on the total amount of protein. Hence, in further embodiments of the present invention, the solution comprising whey protein comprises at most 10% by weight casein or other non-whey protein based on the total amount of protein, preferably at most 5% by weight, more preferably at most 3% by weight casein or other non-whey protein based on the total amount of protein.

In a preferred embodiment of the invention, the whey protein hydrolysate is obtained by using a solution comprising whey protein that is a whey protein concentrate and/or a milk serum protein concentrate. Preferably, the solution comprising whey protein is a whey protein concentrate or milk serum protein concentrate mixed in water.

Whey Protein:

In an aspect of the present invention, the whey protein hydrolysate is obtained by hydrolysing a solution comprising whey protein.

In the context of the present invention, the term “whey protein” pertains to protein that is found in whey or in milk serum. The whey protein present in the solution comprising whey protein may be a subset of protein species found in whey or milk serum or it may be the complete set of protein species found in whey and/or in milk serum. Whey protein is a mixture of proteins isolated from whey, the liquid material created as a by-product of cheese production. Whey proteins are proteins present in the serum phase of either milk or coagulated milk. The proteins of the serum phase of milk are besides from whey proteins also sometimes referred to as milk serum proteins.

The term “milk serum” pertains to the liquid which remains when casein and milk fat globules have been removed from milk, e.g. by microfiltration or large pore ultrafiltration. Milk serum may also be referred to as “ideal whey”.

The term “milk serum protein” or “serum protein” pertains to the protein, which is present in the milk serum.

The term “whey” pertains to the liquid supernatant that is left after the casein of milk has been precipitated and removed. Casein precipitation may e.g. be accomplished by acidification of milk and/or by use of rennet enzyme.

Several types of whey exist such as sweet whey, acid whey and casein whey.

The whey protein present in the whey protein solution of the present invention can be derived from different sources of whey, for example casein whey, acid whey or sweet whey.

In a preferred embodiment of the invention, the whey proteins in the solution comprising whey protein are from sweet whey. Sweet whey comprises primarily the proteins beta-lactoglobulin (BLG) (about 55-65% of total protein content), alpha-lactalbumin (ALA) (about 18-23% of total protein content) and caseinomacropeptide (CMP). However, sweet whey may comprise other proteins, such as immunoglobulins, osteopontin, lactoferrin, bovine serum albumin and fat globule membrane proteins. WPI and WPC may be obtained from sweet whey.

The term “sweet whey” as used herein refers to the liquid remaining after milk has been curdled and strained during the making of rennet type cheeses. “Sweet whey” is obtained during the production of rennet type hard cheese like Cheddar or Swiss cheese. Sweet whey is obtained by adding rennet enzymes to a milk composition, which cleaves kappa-casein into para-kappa-casein and the peptide caseinomacropeptide (CMP), thereby destabilising the casein micelles and causing casein to precipitate. The liquid surrounding the rennet precipitated casein is referred to as sweet whey. The pH value of sweet whey can range between 5.2 and 6.7.

The term “casein whey” (may also sometimes be referred to as sour whey or acid whey) relates to whey, which is obtained from casein/caseinate production. In the context of the present invention, the casein whey is not the same as acid whey.

Casein whey is the whey fraction obtained after separation of casein/caseinates by microfiltration. SPC and SPI are obtained from casein whey.

The term acid whey is used for the whey obtained during the production of acid type cheeses such as cottage cheese and quark. In the preparation of acid type cheeses, casein is removed from milk by acid precipitation, i.e. reducing the pH value of the milk to a pH below 4.6 which is the isoelectric point of casein and which causes the casein micelles to disintegrate and precipitate. The pH is often reduced to a range from 3.8 to 4.6. The liquid surrounding the acid precipitated casein is often referred to as acid whey. WPC and WPI are obtained from acid whey.

In an embodiment of the invention, the whey protein used in the solution comprising whey protein is not acid whey or casein whey.

The whey protein used in the solution comprising whey protein in the present invention may preferably be a whey protein concentrate (WPC) or a milk serum protein concentrate (SPC). Further, a whey protein isolate (WPI) or a milk serum protein isolate (SPI) represent alternatives. The difference between a whey protein concentrate and a whey protein isolate is the composition of the product, particularly the protein content. Whey protein isolates are more pure than concentrates and other non-protein components have been partially removed to “isolate” the whey protein. Thus, a whey protein isolate is having a higher percentage of protein and can be pure enough to be virtually lactose-free, carbohydrate-free, fat-free, and cholesterol-free.

In the present context, the terms “whey protein concentrate (WPC)” and “serum protein concentrate (SPC)” encompass both dry and liquid compositions of whey protein. The protein content in a WPC and SPC used in the present invention is not less than 50% by weight based on the total solid content. However, a whey protein concentrate may comprise higher amounts of whey protein, for example 80% by weight whey protein based on the dry matter content. The dry portion of liquid whey is obtained by the removal of sufficient non-protein constituents from whey so that the dry product comprises not less than 50% by weight whey protein.

A WPC or SPC used in the present invention preferably comprises:
50-89% by weight protein relative to the total solid content
15-70% by weight BLG relative to the total protein content
8-50% by weight ALA relative to the total protein content
0-40% by weight CMP relative to the total protein content.

Alternatively, but also preferred is a WPC or a SPC comprising:
50-89% by weight protein relative to the total solid content
15-80% by weight BLG relative to the total protein content
4-50% by weight ALA relative to the total protein content
0-40% by weight CMP relative to the total protein content.

More preferably, a WPC or a SPC comprises:
70-89% by weight protein relative to the total solid content
30-80% by weight BLG relative to the total protein content
4-35% by weight ALA relative to the total protein content
0-25% by weight CMP relative to the total protein content.

The terms “whey protein isolate” and “serum protein isolate” pertain to dry or liquid compositions, which, generally, are considered almost free of lactose and cholesterol and have a whey protein content of at least 90% by weight based on the total solid content. A whey protein isolate may for example comprise 92% by weight whey protein or higher based on the total solid content. Preferably, the WPI and SPI comprise from 90-100% by weight protein based on the total solid content, such as from 92-99% by weight protein based on the total solid content.

A WPI or a SPI may preferably comprise:
90-100% by weight protein relative to the total solid content
15-70% by weight BLG relative to the total protein content
8-50% by weight ALA relative to the total protein content
0-40% by weight CMP relative to the total protein content.

Alternatively, but also preferred, a WPI or a SPI may comprise:
90-100% by weight protein relative to the total solid content
30-80% by weight BLG relative to the total protein content
4-35% by weight ALA relative to the total protein content
0-25% by weight CMP relative to the total protein content.

Preferred, a WPI may comprise:
90-100% by weight protein relative to the total solid content
60-70% by weight BLG relative to the total protein content
10-25% by weight ALA relative to the total protein content
10-25% by weight CMP relative to the total protein content.

In an embodiment of the invention, the solution comprising whey protein used in the preparation of the whey protein hydrolysate according to the invention comprises a total amount of whey protein in the range of 50-98% by weight of dry matter, such as 60-92% by weight, preferably 70-90% by weight.

Any suitable whey protein source may be used to prepare the solution comprising whey protein according to the present invention. The whey proteins used in solution comprising whey protein according to the present invention are preferably whey proteins from mammalian milk, such as milk from cow, sheep, goat, buffalo, camel, llama, mare, horse and/or deer. In some preferred embodiments of the invention, the whey proteins are derived from bovine (cow's) milk.

It is generally preferred that the content of ash in the solution comprising whey protein is low. With the method of the present invention, it has surprisingly been found that it is possible to obtain a whey protein hydrolysate having a low ash content. Therefore, in an embodiment of the invention, the ash content in the solution comprising whey protein is 6% or less based on the total solid content, more preferably 5.5% or less.

In an embodiment of the invention, the whey protein solution comprises 30% by weight or more of BLG of the total protein content, such as 40% by weight or more of BLG. Most preferably, the whey protein solution comprises 50% by weight or more of BLG based on the total protein content, even more preferably, the whey protein solution comprises BLG in an amount of 55% by weight or more based on the total protein content. In another embodiment of the invention, the whey protein solution comprises BLG in an amount in the range of from 30 to 95% by weight BLG based on total protein content, such as from 40 to 90% by weight BLG based on total protein content, even more preferably from 45 to 80% by weight based on total protein content.

Two-Step Enzymatic Hydrolysis with Heat Treatment:

According to the present invention, the whey protein solution is subjected to a two-step enzymatic hydrolysis with a heat treatment step in between. The first hydrolysis step is step b) of the method and the second hydrolysis step is step e) of the method. Heat treatment is step c) of the method of the present invention.

In step b) of the method of the present invention, a subtilisin from Bacillus licheniformis, a thermolysin from Bacillus amyloliquefaciens and a cysteine endoprotease from Ananas comosus are added to the solution comprising whey protein, and a first hydrolysis step is performed. It is important that a subtilisin from Bacillus licheniformis, a thermolysin from Bacillus amyloliquefaciens and a cysteine endoprotease from Ananas comosus are used in the first hydrolysis step b). Different enzymes have different cleavage sites in the peptides/proteins, and the inventors of the present invention have surprisingly found that the mentioned enzymes in the first hydrolysis step followed by a heat treatment step and a second hydrolysis step will result in a whey protein hydrolysate where the amount of larger peptides (more than 2500 Da) is 7.5% by weight or less. At the same time, the allergenicity is lower than in other partial hydrolysates and the immunogenicity is higher than in extensive hydrolysates produced by ultrafiltration. A subtilisin from Bacillus licheniformis, a thermolysin from Bacillus amyloliquefaciens and a cysteine endoprotease from Ananas comosus have been selected as their presence was required to obtain the desired product with respect to degree of hydrolysis, peptide size distribution, allergenicity and immunogenicity. A similar combination of degree of hydrolysis, peptide size distribution, allergenicity and immunogenicity could not be obtained when these enzymes were not included. Replacing one of the enzymes with for example trypsin did not give the same composition.

The three enzymes can be added simultaneous or separately and the invention should not be limited to any order of adding the three enzymes. Typically, the enzymes are added separately one at the time, but this is only because of technical reasons. The examples in the present patent application shows an embodiment of the invention where the enzymes are added one at the time, but the enzymes could be added simultaneously or in a different order.

The subtilisin from Bacillus licheniformis is typically Alcalase, while the thermolysin from Bacillus amyloliquefaciens typically is Neutrase and the cysteine endoprotease from Ananas comosus is typically Promod 523 MDP. However, other brands of enzymes could be used.

The first hydrolysis step b) is not limited to only comprising the three enzymes and may therefore comprise other enzymes in small amounts. For example, the first hydrolysis step may be performed using three, four, five or more enzymes. Such further enzymes may for example be an endoprotease/peptidase or an exoprotease/peptidase (such as a serine-protease, metallo-protease, cysteine-proteases, aspartic-proteases, amino-peptidases or carboxy-peptidases). One further protease suitable for hydrolysing whey proteins could be a trypsin-like protease of the serine-proteases.

The term “trypsin-like protease” is a protease of microbial origin. Hence, the term “trypsin-like protease”, for example, does not include pancreatin that is not of microbial origin. On the contrary, pancreatin is a mixture of enzymes derived from the pancreas that for example comprises trypsin, chymotrypsin, amylase and lipase. Further, the term “trypsin-like protease” should not be confused with “trypsin”.

In a further embodiment of the invention, the enzymes used for the first hydrolysis step are selected from the following group of enzymes with the Enzyme Commission numbers (EC): EC 3.4.21.62, EC 3.4.24.28, EC 3.4.22.32 and EC 3.4.22.33. However, as mentioned above other enzymes may be present.

After the hydrolysis is initiated in step b), it will be maintained for a period sufficient to obtain the desired degree of hydrolysis. The time-period for the enzymatic hydrolysis, before the hydrolysis is stopped is dependent on the amount and activity of the enzymes used. Preferably, the hydrolysis in the first hydrolysis step is maintained until the degree of hydrolysis is at least 12%, such as at least 15%.

In an embodiment of the present invention, the hydrolysis in step b) is carried out for at least 45 minutes, such as at least 60 minutes, preferably at least 1.5 hours and even more preferably at least 2 hours. Preferably, the hydrolysis in step b) is not limited by an upper time limit and might be carried on until it is desired to stop. However, as an example, the hydrolysis in step b) is carried out for 45 minutes to 8 hours, more preferably 1 hour to 4 hours. In a preferred embodiment of the invention, the hydrolysis in step b) is carried out for 2 to 3 hours.

In another embodiment, the hydrolysis in step b) is carried out at a temperature of 30° C. to 70° C., such as 35° C. to 65° C., preferably 40° C. to 62° C., more preferably, 50° C. to 60° C.

The inventors of the present invention have surprisingly found that the heat-treatment step c) of the hydrolysed composition from step b) before subjecting it to a second hydrolysis step, step e), will result in a whey protein hydrolysate having a high degree of hydrolysis and comprises peptides with a molecular weight of 2500 Da or above in an amount of 7.5% by weight or less of the total amount of peptides.

The heat treatment between the two hydrolysis steps causes any remaining protein derived tertiary structures to unfold. This unfolding leads to exposure of the buried parts of proteins or protein fragments and gives the enzymes in the second step of hydrolysis access to previously inaccessible cleavage sites leading to an increased hydrolysis and thereby a lower fraction of large peptides in the product than when the heat treatment is omitted.

Therefore, in one aspect of the present invention, the hydrolysed composition is after the first hydrolysis step (step b)) and before the second hydrolysis step (step e)) subjected to a heat treatment step (step c)) by adjusting the temperature of the composition to a temperature at at least 60° C. The temperature of at least 60° C. is maintained for a period sufficient to unfold remaining proteins. It is not necessary to heat treat to a temperature where the enzymes are inactivated, but the temperature should be above 60° C. in order to initiate unfolding of remaining protein.

The method of the present invention should not be limited to a specific heating time, because the heating time in step c) is dependent of the type of heating device used and the temperature during the heat treatment. The higher the temperature, the shorter time is necessary for unfolding of the remaining proteins. However, in an embodiment of the present invention, the hydrolysed composition is heat treated in step c) for 5 minutes to 120 minutes, more preferably 10 minutes to 60 minutes. This heating time is relevant if the temperature is 60° C. to 100° C. In another embodiment, the heating time in the heat treatment in step c) is 4 seconds to 10 minutes. This heating time is relevant if the temperature is as high as 90° C. to 145° C. the heat treatment in step c) may be by use of heat exchangers or by use of heat infusion.

Hence, in an aspect of the present invention the heat treatment in step c) is carried out by adjusting the temperature to at least 60° C. In an embodiment, the heat treatment is step c) is carried out by adjusting the temperature to at least 65° C., such as at least 70° C. Typically, the temperature in step c) is in the range of 60° C. to 145° C., more preferably 65° C. to 100° C., such as 68° C. to 90° C. and even more preferably 70° C. to 85° C.

In a preferred embodiment, the heat treatment in step c) is carried out at a temperature of 60° C. to 90° C. for 5 minutes to 2 hours, preferably 70° C. to 80° C. for 5 minutes to 2 hours or for at least 5 minutes. In another embodiment, the heat treatment is carried out by heating to a temperature of 90° C. to 145° C. for 4 seconds to 10 minutes, such as 90° C. to 120° C. for 30 seconds to 10 minutes.

Following the heat treatment in step c), and before initiating the second hydrolysis step, the temperature of the heat treated solution is in step d) adjusted to a temperature suitable for the enzyme(s) used in the second hydrolysis step. If the temperature is too high when step e) is initiated, the enzymes are damaged.

In one embodiment of the present invention, the temperature of the heat treated solution of step c) is adjusted to a temperature of 50° C. to 70° C., more preferably, 50° C. to 60° C.

Following step d), the second hydrolysis step in step e) is initiated by adding at least a subtilisin from Bacillus licheniformis to the hydrolysed composition.

In an embodiment of the present invention, the subtilisin from Bacillus licheniformis is Alcalase.

Other enzymes than subtilisin from Bacillus licheniformis could be added in the second hydrolysis step, but the main enzymes added is subtilisin from Bacillus licheniformis. If other enzymes are added, they are typically added in an amount of 20% or less of the total amount of enzymes, such as 10% or less of the total amount of enzymes.

In an even further embodiment of the present invention, the hydrolysis in step e) is performed with the same time points and temperatures as in step b).

In step f) of the present invention, the enzymatic hydrolysis is stopped by inactivating the enzymes.

The enzymatic hydrolysis is typically stopped in step f) when the degree of hydrolysis (DH) is at least 17%. The degree of hydrolysis (DH) is defined as the percentage of peptide bonds in the original proteins that have been cleaved by hydrolysis. The degree of hydrolysis can be measured as described in Adler-Nissen, J. Determination of the degree of hydrolysis of food protein hydrolysates by trinitrobenzenesulfonic acid. J. Agric. Food Chem. 27, 1256-1262 (1979) and Nielsen, P. M., Petersen, D. & Dambmann, C. Improved method for determining food protein degree of hydrolysis. J. Food Sci. 66, 642-646 (2001).

In a further embodiment, there is essentially no intact protein left when the hydrolysis is stopped in step f). The term “intact protein” refers in the context of the present invention to non-hydrolysed protein. Further, the term “essentially no intact protein” refers in the context of the present invention to that less than 3% of the total protein content is intact protein. Preferably, less than 2% of total protein is intact protein, even more preferably less than 1% of total protein is intact protein.

The method of preparing the whey protein hydrolysate of the present invention should not be limited to the amount of enzyme added during the hydrolysis step, since the amount of enzyme added is dependent on the type of enzyme and the specific activity of the enzyme. However, as a guidance, the enzymatic hydrolysis is performed with a combination of enzymes where the total amount of enzymes is in the range of 0.05 to 10 g per 100 g protein, such as from 0.1 to 7.5 g enzyme per 100 g protein. Preferably, the amount of enzyme is in the amount of 0.2 to 5.0 g per 100 g protein. The term “specific activity” refers to the activity of the enzyme per amount of enzyme.

The solution comprising whey protein should preferably have a pH in the range of 6 to 9 during enzymatic hydrolysis. In a preferred embodiment, the pH during the enzymatic hydrolysis is from 6.0 to 7.5. In this pH range the proteases have the highest specific activity and therefore cleave the proteins to peptides most efficiently. In addition, at this pH range aggregation is avoided, both during the hydrolysis process but also during the heat treatment to inactivate the enzymes.

In step f) of the method of preparing a whey protein hydrolysate according to the present invention, the enzymatic hydrolysis is stopped by inactivating the enzymes. In the context of the present invention, the term “inactivation” refers to irreversible inactivation of the enzyme. The inactivation of enzymes must be irreversible such that the enzymes will not become active under other conditions. The heat treatment in step c) may involve inactivation of the enzymes used in the first hydrolysis step, it depends on the temperature of the heat treatment. However, in step f) all enzymes used in the method, both step b) and step e), are inactivated.

The hydrolysis is stopped when the degree of hydrolysis is at least 17%, such as at least 20%, preferably at least 23%. In an embodiment of the invention, the hydrolysis is stopped in step f) when the degree of hydrolysis is in the range of 17% to 30%, preferably from 19% to 30%, and even more preferably from 21% to 28%.

The inactivation of the enzymes in step f), and hence the stopping of the hydrolysis, can be by any method known in the art. For example, inactivation of the enzymes can be by amending the temperature to a temperature where the enzymes are inactive and denatured. The inactivation and denaturation of the enzymes could also be by amending the pH of the solution to a pH where the enzymes are inactive.

Hence, in an embodiment of the invention, inactivation of the enzymes in step f) is by heating the solution comprising whey protein and added enzymes to a temperature of at least 80° C. The inactivation of enzymes is preferably by heating to a temperature from 80° C. to 130° C., such as from 85° C. to 125° C., even more preferably from 90° C. to 120° C. Inactivation of the enzymes in step f) by heating may for example be by heating to a high temperature for a short time period, such as heating to a temperature from 110° C. to 130° C. for 5 to 30 seconds. Alternatively, the inactivation of the enzymes in step f) may be by heating to a relatively low temperature, but for a longer time period. This could involve heating to from 80° C. to 90° C. for 2 to 10 minutes.

In another embodiment of the present invention, the irreversible inactivation of enzymes in step f) comprises increasing or decreasing the pH of the whey protein solution with added enzymes, i.e. the whey protein hydrolysate to a pH where the enzymes are inactive. In an embodiment of the invention, the pH is increased to a pH of 10 or above. In another embodiment, the pH is decreased to a pH of 4 or below.

In a preferred embodiment of the invention, the method does not comprise any step of ultrafiltration of the whey protein hydrolysate obtained in step f). It was surprising for the inventors of the present invention that adding the heat treatment in step c) between the hydrolysis in step b) and step e) resulted in an amount of peptides having a molecular weight of 2500 Da (or more) of 7.5% by weight or less of the total amount of peptides without reducing the immunogenicity as often seen when applying ultrafiltration.

In the context of the present invention, the term “ultrafiltration” refers to membrane filtration with a membrane having a molecular weight cut off (MWCO) in the range of from 1,500 Da to 50,000 Da, preferably 2,000 Da to 20,000 Da.

During ultrafiltration of protein hydrolysates, fat, intact protein as well as some of the larger peptides are retained by the ultrafiltration membrane and retained in the retentate, while free amino acids, smaller peptides and minerals are in the ultrafiltration permeate.

The whey protein hydrolysate obtained by the method of the present invention may preferably be concentrated and/or dried. Concentration may for example be by one or more of the unit operations nanofiltration, reverse osmosis filtration, and evaporation.

In another embodiment of the invention, the drying step involves one or more of the unit operations spray drying, freeze drying and spin-flash drying, rotary drying and/or fluid bed drying may also be used.

Food products:

In an aspect, the present invention relates to providing a food product comprising the whey protein hydrolysate according to the invention.

In preferred embodiments, the food product is an infant nutritional product, such as an infant formula or other infant nutritional products.

If the food product is in a liquid form, the food product may comprise the whey protein hydrolysate according to the invention. Preferably the food product comprises the whey protein hydrolysate in an amount corresponding to 2 to 25% by weight hydrolysed whey protein, preferably from 3 to 20% by weight hydrolysed whey protein, such as from 3 to 15% by weight hydrolysed whey protein, even more preferably form 3 to 10% by weight hydrolysed whey protein.

In a preferred embodiment of the invention, a liquid food product comprising the whey protein hydrolysate of the invention has a neutral pH, i.e. a pH in the range from 6.5 to 8.0 in a 4% protein solution at 22° C.

The whey protein hydrolysate according to the present invention may be used in infant nutrition, such as in infant formulas. In the context of the present invention, the term “infant formula” refers to any type of infant formulas, including follow-on formula, growing-up formula and preterm formula.

If the whey protein hydrolysate according to the present invention is used in an infant formula, the protein content in the infant formula is in the range of 1.6 to 5.0 g/100 kcal. Besides from whey protein hydrolysate, the infant formula may also comprise lipids, vitamins and minerals as well as carbohydrates, such as lactose and oligosaccharides.

The whey protein hydrolysate according to the present invention may also be used in the preparation of other infant nutrition than infant formulas, for example in smoothies, porridges and the like.

The whey protein hydrolysate of the invention is preferably intended to be used in infant formula for infants, more preferred infants at risk of developing an allergy. However, it is not excluded that the hydrolysate may be used for other purposes such as medical nutrition applications.

Hence, in still another embodiment of the invention, the whey protein hydrolysate is used as a food ingredient in the preparation of clinical beverages. In the context of the present invention, the term “clinical beverage” refers to beverages having a clinical or medical indication. For example, a clinical beverage could have a health related effect. A clinical beverage is typically used by hospitalized or elderly people with nutritional difficulties or individuals that require pre-digested proteins in order to recover from a medical condition. For example, a clinical beverage could be a beverage used for individuals suffering from malnutrition or malabsorption. The clinical beverage could also be for individuals suffering from gastro-intestinal diseases. In the context of the present context, the term “clinical beverage” and medical beverage” have the same meaning.

A clinical beverage could for example comprise the whey protein hydrolysate of the invention in an amount corresponding to the beverage comprising from 2 to 20% by weight hydrolysed whey protein. The clinical beverage may comprise carbohydrates in an amount of from 5 to 50% by weight of the beverage. The amount of carbohydrates may for example be in the range of from 10 to 40% by weight, such as from 15 to 35% by weight. The clinical beverage may also comprise fat. For example, the fat content in the clinical beverage could be in the range of from 2 to 30% by weight, such as from 3 to 20% by weight, more preferably from 3 to 18% by weight.

In an example, the clinical beverage comprises the whey protein hydrolysate according to the invention corresponding to an amount of 4-10% by weight hydrolysed whey protein, 3-15% by weight fat and 10-35% by weight carbohydrates.

The clinical beverage preferably has a neutral pH value, i.e. a pH in the range of 6.5 to 8.0.

The clinical beverage may be in the form of a clear beverage, a milky beverage, a tube feed, or in the form of a powder to be reconstituted in a liquid.

In an embodiment, the whey protein hydrolysate of the invention may also be used in the preparation of a juice-style beverage. The juice-style beverage preferably comprises the whey protein hydrolysate according to the invention in an amount corresponding to 4-10% by weight hydrolysed whey protein, 0-1% by weight fat and 15-35% by weight carbohydrates.

If the clinical beverage is in the form of a tube feed, it may comprise the whey protein hydrolysate according to the invention in an amount corresponding to 4 to 15% by weight hydrolysed whey protein, about 5-35% by weight carbohydrates and about 3 to 15% by weight fat.

In an embodiment of the present invention, the whey protein hydrolysate may be used for reducing the risk of allergies to cow's milk proteins in infants.

In an embodiment of the present invention, the whey protein hydrolysate may be used for reducing the risk of allergies to cow's milk proteins in patients in need thereof.

Hence, the present invention relates to the whey protein hydrolysate according to the invention, for reducing the risk of allergies to cow's milk proteins in infants or patients in need thereof.

Further, the present invention relates to the whey protein hydrolysate according to the invention, for prevention of or reducing the risk of developing atopic dermatitis, asthma and/or allergic rhinitis in infants or patients in need thereof.

The term “atopic dermatitis” may also be referred to as eczema, while allergic rhinitis may be referred to as hay fever.

It should be noted that embodiments and features described in the context of one of the aspects of the present invention also apply to the other aspects of the invention.

All patent and non-patent references cited in the present application are hereby incorporated by reference in their entirety.

The invention will now be described in further details in the following non-limiting examples.

### EXAMPLES

### Example 1: Methods of analysis

### Example 1.1: Determination of Degree of Hydrolysis (DH)

The degree of hydrolysis (DH) is defined as the percentage of peptide bonds cleaved by hydrolysis, see equation (1) below. The DH value gives information about the number of peptides formed, which is related to the number of available peptide bonds.

The DH of the whey protein hydrolysate was measured as described in Adler-Nissen, J. Determination of the degree of hydrolysis of food protein hydrolysates by trinitrobenzenesulfonic acid. J. Agric. Food Chem. 27, 1256-1262 (1979) and Nielsen, P. M., Petersen, D. & Dambmann, C. Improved method for determining food protein degree of hydrolysis. J. Food Sci. 66, 642-646 (2001). In equation (1), h represents the number of cleaved peptide bonds and hₜₒₜₐₗ represents the total number of peptide bonds available. Thus, DH gives the percentage of cleaved peptide bonds.

DH=(number of free amino terminals)/(total number of available peptide bonds)·100%=h/hₜₒₜₐₗ·100% Equation (1):

The free alpha-amino groups formed after hydrolysis react with o-phthalaldehyde (OPA) and form a yellow complex which absorbs light at 340 nm and therefore can be measured spectrophotometrically. Based on the color formation, DH can be calculated.

Hydrolysates were resuspended in water at appropriate concentrations (0.03-0.08% protein) and 2 volumes were reacted for 2 minutes at 25° C. with 15 volumes of OPA reagent (100 mM Na₂B₄O₇, 0.1% sodiumdodecyl-sulphate, 6 mM DL-dithioreitol, 6 mM o-phtalaldehyde and 2% ethyl alcohol). Similar reactions were made to create concentration series of L-serine. Next, the absorbance at 340 nm (A340) was measured and the A340 signal from the reaction of OPA with water was subtracted. To find the true DH, the serine equivalents measured in the supernatants were corrected as suggested by Adler-Nissen for the Trinitrobenzenesulfonic acid method [Adler-Nissen J; Agricultural and Food Chemistry, 1979 27 (6) 1256] which gave the same response as the described OPA method. The factors used for the whey protein hydrolysates were a=1, b=0.4, hₜₒₜₐₗ=8.8.

### Example 1.2: Determination of Total Protein

The total protein content (protein equivalents) of a sample is determined by:
1) Determining the total nitrogen of the sample following ISO 8968-1/2IIDF 020-1/2-Milk—Determination of nitrogen content—Part 172: Determination of nitrogen content using the Kjeldahl method.
2) Calculating the total amount of protein as: Nx6.38

### Example 1.3: Method to Determine the Peptide Distribution in the Whey Protein Hydrolysates

Size exclusion chromatography (SEC) was used to analyze the molecular weight distribution of the peptides in the whey protein hydrolysate. SEC is used to separate polymer type molecules by size. A mixture of components of different size, here peptides, can be separated by SEC. The elution time is dependent on the size of the molecule. The smaller the molecule, the longer the elution time.

The samples were dissolved in the mobile phase to a concentration of 0.5% w/v. Before injection, the sample was filtered through a 0.45 μm filter. Chromatographic separation was performed on three TSK G2000 SWXL (125 Å, 5 μm, 7.5 mm×300 mm) columns bound in series. A buffer of 0.0375 M phosphate buffer, 0.375 M ammonium chloride, 0.1% trifluoroacetic acid (TFA), and 25% acetonitrile (CH₃CN) was used as the mobile phase with a flow of 0.7 mL per minute. Detection of the peptides was performed using a UV-detector measuring at 214 nm. Based on the retention time, the distribution of peptides is divided according to size, and the relative amount is given according to the molecular weight.

### Example 2—Preparation of the Whey Protein Hydrolysate of the Invention

Example 2 shows an example of preparing the whey protein hydrolysate according to the present invention.

As feed material and substrate, a solution of Lacprodan® DI-8306, a whey protein concentrate (WPC) from Arla Foods Ingredients was used. The WPC was diluted in water to a protein concentration of 8% by weight. The solution was heated to approximately 50° C. and pH adjusted to 7.0 using a solution of 4.2% KOH/5.8% NaOH. Neutrase conc BG from Novozymes A/S was added in an amount of 16 AU-N/kg protein (AU-N refers to Anson Units-Neutrase) to initiate the first hydrolysis step while maintaining pH at 7 using a mixture of sodium hydroxide and potassium hydroxide. After about 15 minutes, Promod 523 MPD from Biocatalysts was added in an amount of 12500 GDU/kg protein (GDU refers to Gelatin Degrading Units) and the hydrolysis continued while maintaining pH at 7. After another 15 minutes (30 minutes from starting the hydrolysis), Alcalase conc BG from Novozymes A/S was added in an amount of 14 AU-A/kg protein (AU-A refers to Anson Units-Alcalase) while maintaining pH at 7 until a total mass (kg) of 4.2% KOH/5.8% NaOH corresponding to 27% of the protein mass was added (including the amounts used for initial pH adjustments) and the hydrolysis continued for another 75 minutes before the temperature of the hydrolysed solution was adjusted to 75° C. The heat-treatment at 75° C. was carried out for 30 minutes before the temperature was adjusted to 65° C. and a second hydrolysis step was performed by adding Alcalase conc BG in an amount of 35 AU-A/kg protein. Hydrolysis was continued for 120 minutes followed by addition of 10% citric acid in an amount (kg) corresponding to 7.3% of the amount of protein (kg) in the reaction.

After 120 minutes of hydrolysis in the second hydrolysis step, the hydrolysis was stopped by heating to 90° C. with a holding time of 240 seconds to inactivate the enzymes. The whey protein hydrolysate obtained was subjected to reverse osmosis to remove water, and subsequently pasteurized before spray drying and collection of the final powder.

### Example 3—Analysis of the Whey Protein Hydrolysate of the Invention

The peptide distribution of the whey protein hydrolysate of the present invention has been measured and compared to the peptide distribution of the peptides in the commercially available infant formula composition NAN HA1 from Nestle. NAN HA1 is an infant formula known to be low-allergenic and is promoted for healthy children at risk of developing allergy towards milk proteins. The protein source in NAN HA1 formula is a protein hydrolysate. NAN HA1 has been tested in many clinical studies, and has been shown to be effective in preventing allergic reactions/manifestations in infants at risk of developing allergy. However, as shown in table 1 below, the peptide distribution of NAN HA1 is very different from the whey protein hydrolysate of the present invention.

A sample of the whey protein hydrolysate of the invention, prepared as described in example 2, and a sample of NAN HA1 were prepared and the peptide size distribution was measured by the method disclosed in example 1.3. The result is shown in table 1. The sample of the whey protein hydrolysate of the invention is referred to as “WPHᵢₙᵥₑₙₜᵢₒₙ”.

TABLE 1

| | <375 | 375-750 | 750- | 1250- | >2500 |
|---|---|---|---|---|---|
| | Da | Da | 1250 Da | 2500 Da | Da |
| WPHᵢₙᵥₑₙₜᵢₒₙ | 17 | 37.5 | 25.5 | 15.5 | 4.5 |
| NAN HA1 | 9.3 | 21.4 | 17.2 | 27.2 | 24.9 |

As shown in Table 1, the peptide size distribution in the whey protein hydrolysate of the present invention is very different from the peptide size distribution of the peptides in NAN HA1. For example, the amount of larger peptides in the whey protein hydrolysate of the invention is lower (4.5% of the peptides have a molecular weight above 2500 Da) than in NAN HA1 (24.9% of the peptides in NAN HA1 have a molecular weight of more than 2500 Da). Further, the amount of small peptides below 375 Da in the whey protein hydrolysate of the invention is higher (17%) than in NAN HA1 (9.3%).

Further, the degree of hydrolysis (DH) and ash content of the whey protein hydrolysate of the invention were determined. The DH was determined according to the method disclosed in example 1.1, while the ash content was determined at Eurofins Laboratories using the gravimetric method according to the GB.5009.4 standard. The results are shown in table 2.

TABLE 2

| | |
|---|---|
| Ash (weight percent of solid content) | 4.8 |
| DH (%) | 23.5 |

It is assumed that the DH of the hydrolysate used in NAN HA1 is lower than in the hydrolysate of the present invention, since the amount of larger peptides in NAN HA1 is higher than in the hydrolysate of the present invention. For example, about 51.6% of the peptides in NAN HA1 has a molecular weight of 1250 Da or higher, where only 20% of the peptides in the whey protein hydrolysate of the present invention has a molecular weight of 1250 Da or higher.

The ash content of the whey protein hydrolysate of the invention was about 4.8% by weight of the solid content and 4.5% by weight in the whey protein hydrolysate in powder form (comprising 5-6% moisture). The amount of ash in the whey protein hydrolysate was found to be surprisingly low as compared to other similar whey protein hydrolysates.

### Example 4—Analysing Presence of Intact Protein

The amount of the whey proteins alpha-lactalbumin and beta-lactoglobulin in the whey protein hydrolysate of the invention, WPHᵢₙᵥₑₙₜᵢₒₙ, was measured using a HPLC.

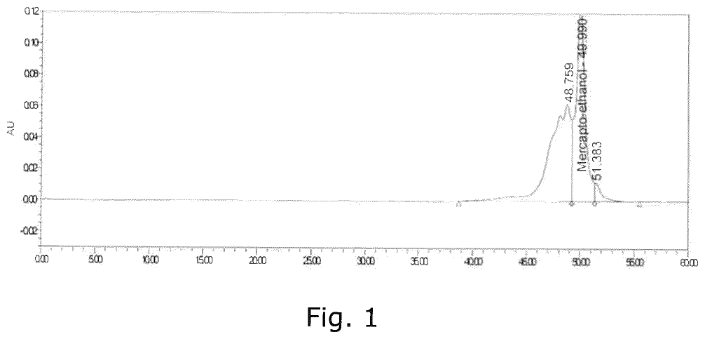

The samples were dissolved in 6M guanidine HCl buffer with 2-mercapthoethanol used as reducing agent. The separation is based on size exclusion chromatography (SEC) of denaturated proteins. The method uses 6M Guanidine HCl buffer as both sample solvent and HPLC mobile phase. The separation is based on two TSK-GEL G3000SWXL (7.7 mm×30.0 cm) columns and a guard column in series to achieve adequate separation of the major proteins in raw materials. The detection and quantification of alpha-lactalbumin performed by UV detection (280 nm). Beta-lactoglobulin will elute after 33.50 minutes while Alpha-lactalbumin will elute after 35.8 minutes. The result of the HPLC analysis is shown in FIG. 1, where it is clear that no detectable beta-lactoglobulin or alpha-lactalbumin is present in the WPHᵢₙᵥₑₙₜᵢₒₙ.

### Example 5—Analysis of Allergenic Potential by ELISA

The allergenic potential of the whey protein hydrolysate of the present invention was measured by Intertek Food Services GmbH using the Enzyme-Linked Immunosorbent Assay (ELISA) testing method, R4901. The R4901 assay is a competitive enzyme immunoassay that quantitatively determines the content of beta-lactoglobulin (BLG) in hydrolysed milk products or baby food. The assay kit is known as RIDASCREEN beta-lactoglobulin, and is available from R-Biopharm AG (article number: R4901). The microtiter wells are coated with the BLG and standards, sample solutions and anti-BLG are added. Free and immobilized BLG compete for the antibody binding sites. After washing, secondary antibodies labelled with peroxidase are added and bind to the antibody-BLG-complexes. Any unbound enzyme conjugate is then removed by a washing step. Chromogenic enzyme substrate is added to the wells. Bound enzyme conjugate converts the colourless chromogenic substrate into a coloured product. The measurement is made photometrically and the absorption is inversely proportional to the BLG concentration in the sample.

A sample of the following was analysed:
WPHᵢₙᵥₑₙₜᵢₒₙ—the whey protein hydrolysate obtained in example 2
NAN HA1
Peptigen® IF-3080 from Arla Foods Ingredients (an ultrafiltered whey protein hydrolysate having a DH of 23-29%)

By the terms “n=3” and “n=4” are meant that 3 and 4 samples respectively were tested. The result is shown in table 3.

Further, the amount of intact non-hydrolysed protein in the raw material used for preparing the whey protein hydrolysate can be calculated and thus the fold reduction of the beta-lactoglobulin (BLG) content may be calculated. The whey protein hydrolysate according to the invention is prepared by using a whey protein concentrate comprising 54% BLG, i.e. 540,000 mg BLG/kg powder. The protein content in the powder is 80% by weight and therefore the amount of BLG corresponds to 432,000 mg BLG/kg protein. The fold reduction of BLG is also shown in table 3.

TABLE 3

| | | Ratio between BLG in |
|---|---|---|
| | | non-hydrolysed substrate |
| | mg BLG/kg protein | and BLG in WPHᵢₙᵥₑₙₜᵢₒₙ |
| WPHᵢₙᵥₑₙₜᵢₒₙ (n = 3) | <300 | >1440 (*) |
| NAN HA (n = 4) | <400 | |
| Peptigen IF-3080 | <20 | |

(*) The ratio between intact non-hydrolysed BLG and BLG in WPHᵢₙᵥₑₙₜᵢₒₙ was calculated as (432000:300 = 1440)

As shown in table 3, the content of BLG in the whey protein hydrolysate of the invention has been reduced by at least a 1440 fold. The BLG content and hence the allergenic potential of WPHᵢₙᵥₑₙₜᵢₒₙ is similar to the allergenic potential of NAN HA1 and as expected the allergenic potential is also low for the ultrafiltered hydrolysate Peptigen® IF-3080. By ultrafiltering the hydrolysate, larger peptides are removed. Since the allergenic potential of NAN HA1 is known from clinical studies to be low, the allergenic potential of WPHᵢₙᵥₑₙₜᵢₒₙ is also expected to be as low as in NAN HA1 as the BLG content in the two products is similar. Hence, the protein hydrolysate of the invention and NAN HA1 have a similar and low allergenic potential.

### Example 6—Analysis of Degranulation

Degranulation of the whey protein hydrolysate of the invention as a measurement of allergenicity was analysed.

The degranulation and hence allergenicity was measured by Polpharma Biologics by use of an RBL cell assay. RBL (rat basophil leukemia)-cells were sensitized with a pool of six different chimeric (mice/human) antibodies directed against beta-lactoglobulin. When exposed to different hydrolysates, degranulation of the cells was measured (by measuring extracellular hexosaminidase activity) and allegenicity determined.

Degranulation refers to the binding of epitopes from the hydrolysates to the antibodies resulting in crosslinking of antibodies, release of mediators, and finally allergic response.

A result of no or low degranulation is an indication of reduced binding and cross-linking of epitopes from hydrolysates to antibodies, as the epitopes have been destroyed/reduced in the hydrolysis process.

The degranulation effect of the whey protein hydrolysate of the invention (WPHᵢₙᵥₑₙₜᵢₒₙ) was analysed and allergenicity determined, as well as the commercially available infant formula NAN HA1 comprising hydrolysed proteins.
Sample 1-3: NAN HA1
Sample 4-6: WPHᵢₙᵥₑₙₜᵢₒₙ

The results are shown in table 4 below:

TABLE 4

| Hydrolysate | Product type | mg BLG/kg protein |
|---|---|---|
| Sample 1 | NAN HA1 | 83 × 10³ |
| Sample 2 | NAN HA1 | 84 × 10³ |
| Sample 3 | NAN HA1 | 88 × 10³ |
| Sample 4 | WPHᵢₙᵥₑₙₜᵢₒₙ | 309 |
| Sample 5 | WPHᵢₙᵥₑₙₜᵢₒₙ | 378 |
| Sample 6 | WPHᵢₙᵥₑₙₜᵢₒₙ | 441 |

Hence, the data in table 4 shows that the allergenicity of the whey protein hydrolysate of the invention is lower than the allergenicity of NAN HA1, i.e. indicating that the crosslinking potential of antibodies to epitopes is lower when using the hydrolysate of the invention than when using NAN HA1. The allergenicity of the WPHᵢₙᵥₑₙₜᵢₒₙ is below 500 mg BLG/kg protein. On the contrary, the allergenicity measured by degranulation of NAN HA1 is higher than 80×10 3 mg BLG/kg protein. Thus, even though it has been shown that NAN HA1 is not allergenic in clinical studies, the allergenicity (degranulation) is more than 150 fold higher than in the whey protein hydrolysate of the present invention.

### Example 7—Analysis of Immunogenicity and Allergenicity/Sensitization

Immunogenicity and allergenicity/sensitization of the whey protein hydrolysate of the invention was compared to a whey protein concentrate and an extensively hydrolysed, filtered whey protein product using a Brown Norway rat model for food allergy sensitization. As sensitization is a precursor of allergy, it will be perceived as a marker of allergenicity.

The Brown Norway rats are high IgE responders, making them suitable to resemble atopic humans in their predisposition to develop allergy. The experiments using the Brown Norway rat model were conducted by the Technical University of Denmark. The Brown Norway rat model is described in the article “Characterization of the Immunogenicity and Allergenicity of Two Cow's Milk Hydrolysates—A Study in Brown Norway Rats”, Bøgh et al., published in Scandinavian Journal of Immunology, 23 Dec. 2014, DOI:10.1111/sji.12271.

To evaluate the immunogenicity and sensitization, the rats were intraperitoneal injected with either 100 μg or 200 μg per dose of a sample/product and the specific IgG1 and specific IgE antibodies were measured using ELISA assays as disclosed in “Preclinical Brown Norway Rat Models for the Assessment of Infant Formulas in the Prevention and treatment of Cow's Milk Allergy”, Jensen et al., published in International Archives of Allergy and Immunology, 13 Feb. 2019, DOI: 10.1159/000495801 and Bogh et al, 2014. The dose of 200 μg was used to ensure maximum sensitization—if possible—while the dose of 100 μg was used to investigate the difference in the sensitization of the products. The immunogenicity and sensitization of the products were evaluated based on the level of specific IgG1 (immunogenicity) and specific IgE (sensitization/allergenicity) antibodies towards the intact whey product WPC Lacprodan® DI-8306 from Arla Foods Ingredients.

The rats were maintained on a milk-free diet for a least 10 generations and thus naive to cow's milk. The animals were between 5-8 weeks old, had water and fed ad libitum and stored in same-gender pairs of three in macrolon cages. Other conditions for the rats are disclosed in Jensen et al., 2019 and Bogh et al., 2014.

The rats were i.p. immunised three times during the study; at day 0, day 14 and at day 28 with a solution of 0.5 mL/animal. Blood samples were taken from the tongue vein at day 21 and day 28, in addition to being collected when the animals were sacrificed on day 35. All blood samples were collected and converted into sera as disclosed in Jensen et al., 2019 and Bogh et al., 2014 to obtain sera. The sera were analysed for specific IgG1 (immunogenicity) and specific IgE (sensitization) using two different ELISAs as disclosed in Jensen et al., 2019 and Bogh et al., 2014.

The immunogenicity (IgG1) and sensitization (IgE) of the following samples were tested:
PBS (phosphate-buffered saline).
WPC: intact whey protein from the whey protein concentrate Lacprodan® DI-8306 from Arla Foods Ingredients was used.
Extensively hydrolyzed WPC1: the extensively, filtered hydrolysed whey protein, Peptigen® IF-3080 (batch 1) from Arla Foods Ingredients was used having a degree of hydrolysis of 25-30.
Extensively hydrolyzed WPC2: another extensively, filtered hydrolysed whey protein hydrolysate was used, Peptigen® IF-3080 (batch 2) having a degree of hydrolysis of 25-30.
Partially hydrolyzed WPC1: a whey protein hydrolysate that has been prepared by the first step of the method of the invention, but without any heat treatment and the secondary hydrolysis.
Partially hydrolyzed WPC2: the whey protein hydrolysate of the invention prepared according to the method of example 2. The partially hydrolyzed WPC2 corresponds to the WPHᵢₙᵥₑₙₜᵢₒₙ in earlier examples and has a degree of hydrolysis of approximately 23.5.

All samples were dissolved in PBS and injected i.p, to the rats.

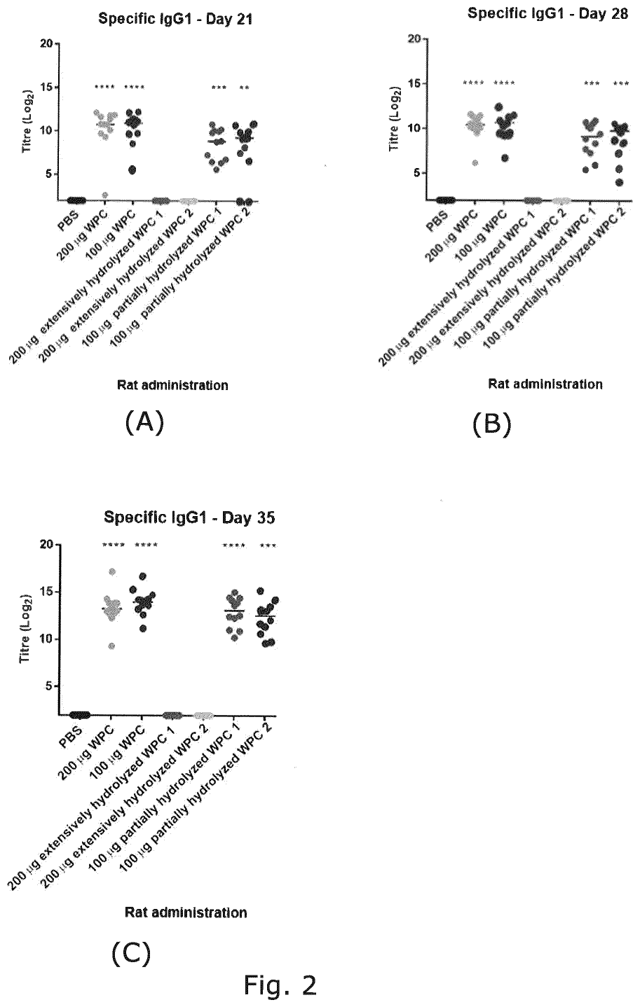

FIGS. 2A, B and C show the IgG1 titre in the rat sera on day 21, 28 and 35 respectively. Statistically significant differences between the different groups compared to the control group dosed with PBS are shown with asterisk: *p≤0.05, **p≤0.01, ***p≤0.001, **** p≤0.0001. The statistical analyses were made with GraphPad Prism version 7.00 (San Diego, CA, USA). A) shows specific IgG1 one week after the second dosing (Day 21), B) shows specific IgG1 two weeks after the second dosing (Day 28), C) shows specific IgG1 one week after the third dosing (Day 35).

From FIG. 2A-C, it is clear that after immunisation, the group immunised with the whey protein hydrolysate of the invention and the whey protein concentrate, all developed significantly higher levels of specific IgG1 against Lacprodan® DI-8306 as compared to the control group indicating that these products induced immunogenic IgG response. On the contrary, in the group of rats injected with the extensively hydrolysed whey protein, there were no detection of IgG in sera indicating that the extensively hydrolysed whey protein hydrolysates did not induce an immunogenic IgG response.

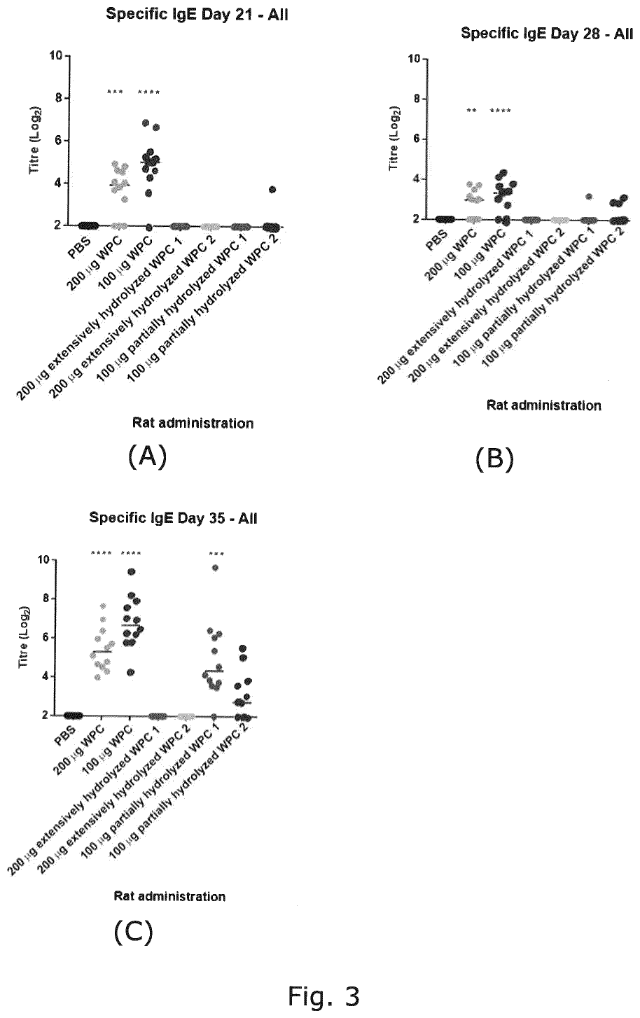

FIGS. 3A, B and C show the sensitization as the titre of specific IgE towards the WPC Lacprodan® DI-8306 on day 21, 28 and 35 respectively. Statistically significant differences between the different groups are shown with asterisk: *p≤0.05, **p≤0.01, ***p≤0.001, **** p≤0.0001. A) shows specific IgE one week after the second dosing (Day 21), B) shows specific IgE two weeks after the second dosing (Day 28), C) shows specific IgE one week after the third dosing (Day 35).

FIG. 3 shows that the IgE titre after 28 days is higher in animals immunised by WPC than animals immunised by extensively hydrolysed WPC1 and WPC2 or partially hydrolysed WPC 1 or WPC 2. Hence, the sensitization of the whey protein hydrolysate of the present invention and the extensively hydrolysed whey protein hydrolysates are low. After 35 days, the titre of partially hydrolysed WPCs are higher than the titre of extensively hydrolysed WPCs. The titre of partially hydrolysed WPC 2 of the invention is lower than the titre of WPCs after 35 days.

1. A whey protein hydrolysate composition having the following characteristics:
i) comprising peptides with a molecular weight of 2500 Da or above in an amount of 7.5% by weight of the total protein content or less;
ii) an allergenicity corresponding to a concentration of beta-lactoglobulin (BLG) being less than 500 mg BLG/kg protein as measured in an RBL cell assay;
iii) an immunogenicity based on the serum IgG titre of immunised animals that is essentially the same as for non-hydrolysed whey protein;
iv) comprising essentially no intact protein, and wherein the degree of hydrolysis is at least 17%.

2-14. (canceled)

15. The whey protein hydrolysate according to claim 1, wherein the degree of hydrolysis is in the range of 17% to 30%.

16. The whey protein hydrolysate according to claim 1, wherein the concentration of beta-lactoglobulin is less than 500 mg/kg of the total protein measured using an ELISA assay.

17. The whey protein hydrolysate according to claim 1, wherein the whey protein hydrolysate comprises an amount of ash in the range of 6.0% by weight or less of the solid content.

18. A method of preparing a whey protein hydrolysate comprising:
a) providing a solution comprising whey protein;
b) adding at least a subtilisin from Bacillus licheniformis, a thermolysin from Bacillus amyloliquefaciens and a cysteine endoprotease from Ananas comosus to the solution comprising whey protein and performing a first hydrolysis step;
c) heat-treating the hydrolysed solution of b) by adjusting the temperature to at least 60° C. and maintaining the temperature at least at 60° C. for a time sufficient to unfold remaining folded protein;
d) adjusting the temperature of the heat-treated solution of c) to a temperature of 50° C. to 70° C.;
e) adding at least subtilisin from Bacillus licheniformis to the hydrolysate of d) and performing a second hydrolysis step; and
f) inactivating the enzymes when the degree of hydrolysis is at least 17% to obtain a whey protein hydrolysate.

19. The method according to claim 18, wherein the method comprises no ultrafiltration of the obtained whey protein hydrolysate.

20. The method according to claim 18, wherein the first hydrolysis in b) is for a period of at least 45 min.

21. The method according to claim 18, wherein the pH is 6.0 to 7.5.

22. A food product comprising the whey protein hydrolysate according to claim 1.

23. The food product according to claim 22, wherein the food product is an infant nutritional product.

24. A method of reducing an allergy to a bovine milk protein in a subject comprising providing the whey protein hydrolysate according to claim 1 to a subject in need thereof.

25. A method of inhibiting atopic dermatitis, asthma, or allergic rhinitis in a subject comprising providing the whey protein hydrolysate of claim 1 to a subject in need thereof.
